(19) United States
(12) Patent Application Publication
(10) Pub. No.: US 2014/0051927 A1
(43) Pub. Date: Feb. 20, 2014

(54) ELECTRONIC CABLE ASSEMBLIES FOR USE WITH MEDICAL DEVICES

(71) Applicant: Boston Scientific Scimed, Inc., Maple Grove, MN (US)

(72) Inventors: Russell DRESHER, Hudson, MA (US); Paul AQUILINO, Walpole, MA (US); Rachael RHEAUME, Framingham, MA (US)

(73) Assignee: Boston Scientific Scimed, Inc., Maple Grove, MN (US)

(21) Appl. No.: 13/957,942

(22) Filed: Aug. 2, 2013

Related U.S. Application Data
(60) Provisional application No. 61/684,862, filed on Aug. 20, 2012.

(51) Int. Cl. H01B 7/02; A61B 1/05; A61B 1/12; A61B 1/00

(52) U.S. Cl. CPC H01B 7/02; A61B 1/00114; A61B 1/05; A61B 1/12; A61B 1/00103
USPC 600/110; 174/116; 600/132

(57) ABSTRACT

The present disclosure is directed to a cable assembly for a medical device. The cable assembly may include a plurality of conductors each defining a longitudinal axis. The plurality of conductors may be spaced so that the longitudinal axes of the plurality of conductors are parallel. The cable assembly may further include a layer of insulation material completely surrounding each of the plurality of conductors. The layer of insulation material may also extend between adjacent conductors to substantially fill areas between adjacent conductors along a length of the cable assembly, and at least one conductor may be electrically coupled to electronics at a distal portion of the electronic device.

### CROSS-REFERENCE TO RELATED APPLICATIONS

This application claims the benefit of priority from U.S. Provisional Application No. 61/684,862, filed on Aug. 20, 2012, the entirety of which is incorporated by reference herein.

### FIELD

Embodiments of the disclosure relate generally to medical devices and systems. More particularly, embodiments of the disclosure relate to electronic cable assemblies for use with medical devices, including, for example, endoscopes of medical imaging systems.

### BACKGROUND

An endoscope is a flexible medical device introduced into the body for diagnostic or therapeutic purposes. Typically, an endoscope, and like devices such as a colonoscope and a ureteroscope, are inserted into the body through an opening (a natural opening or an incision), and are delivered to a work site inside the body through a body cavity. Generally, such devices include a steerable flexible tube with one or more lumens to deliver, for example, therapy, visualization, illumination, irrigation, or aspiration. These devices also include an illumination device disposed in the illumination lumen to illuminate a field of view at the worksite, and an imaging device disposed in the visualization lumen to allow a surgeon to see the worksite. The overall diameter of the endoscope generally depends on the anatomical lumen or organ being observed.

In procedures such as, for example, cholangioscopy or cytoscopy, the diameter of the endoscope may be sufficiently small to ensure access to bile ducts and tight strictures. As a result, the wall between the visualization lumen and the irrigation and/or aspiration lumens may be thin and, consequently, may be prone to processing defects and/or mechanical damage during a medical procedure. These defects may allow fluid from the irrigation and/or aspiration lumen to enter into the visualization lumen. Where an electronic imaging endoscope is used, fluid may surround an electrical cable disposed in the visualization lumen and fill an area formed between adjacent conductors of the electronic cable assembly. This may result in video degradation or failure.

In order to avoid such issues, the camera may be designed to drive a larger capacitive load. This, however, may require a camera and an endoscope of a larger physical size which, in turn, may reduce the ability of the endoscope to access and visualize tight strictures. The alternatives, such as increasing the thickness of the visualization and/or irrigation lumen walls or using micro-coaxial cables may either increase the size of the endoscope or the manufacturing expense. While the cost may be less of an issue with reusable endoscopes where the cost can be amortized due to repeated usage, this cost may be prohibitively high in the case of disposable endoscopes. Thus, it may be desirable to have alternative electronic cable assemblies that prevent video degradation and are relatively inexpensive to manufacture.

### SUMMARY

Embodiments of the present disclosure are directed to a medical imaging system having a medical device, such as, for example, an endoscope capable of producing an image signal in an analog or digital video format. The endoscopes used with the system of the present disclosure may be reusable or single-use devices that are sufficiently inexpensive to manufacture such that the endoscope can be considered a single-use, disposable item. The electronic cable assemblies used with the system of the present disclosure may be used in disposable endoscopes, endoscopes having small diameter lumens, and/or endoscopes having thin-wall lumens. In addition, though much of the disclosure focuses on the use of the cable embodiment in combination with an endoscope, the cable embodiment may be used with other medical devices, as will be discussed below.

One embodiment of the present disclosure is directed to a cable assembly for a medical device. The cable assembly may include a plurality of conductors each defining a longitudinal axis. The plurality of conductors may be spaced so that the longitudinal axes of the plurality of conductors are parallel. The cable assembly may further include a layer of insulation material completely surrounding each of the plurality of conductors and extending between adjacent conductors to substantially fill areas between adjacent conductors along a length of the cable assembly and in at least one conductor may be electrically coupled to electronics at a distal portion of the electronic medical device.

In various embodiments, the cable assembly may include one or more of the following additional features: wherein the layer of insulation material completely fills areas between adjacent conductors along the length of the cable assembly; wherein the layer of insulation material is arranged to exclude fluid from being introduced between the plurality of conductors when the cable assembly is in a fluidic environment; wherein the longitudinal axes of the plurality of conductors are aligned in a single plane; and wherein a portion of at least one conductor is exposed.

Another embodiment of the present disclosure is directed to a medical device. The medical device may include an elongate member having a distal end, a first lumen, and a second lumen, The medical device may further include a fluid port defined in the distal end of the elongate member. The fluid port may be in communication with the first lumen for delivering fluid. The medical device may also include an imaging device at the distal end of the elongate member and a cable assembly electrically coupled to the imaging device and extending proximally in the second lumen. The cable assembly may include two or more conductors and a single layer of insulation. The insulation may substantially fill areas between adjacent conductors.

In various embodiments, the medical device may include one or more of the following additional features: wherein the single layer of insulation completely surrounds each conductor of the two or more conductors; wherein the single layer of insulation material is arranged to exclude fluid from being introduced between the two or more conductors when the cable assembly is in a fluidic environment; wherein the longitudinal axes of the two or more conductors are parallel and disposed in a single plane; wherein the cable assembly has a length, and wherein each of the two or more conductors is arranged longitudinally along the length of the cable assembly; wherein the two or more conductors have a constant pitch along the length of the cable assembly; wherein the conductors are fine gage conductors; wherein the imaging device is configured to produce an image signal in an analog format; and wherein the single layer of insulation material completely fills areas between adjacent conductors.

Another embodiment of the present disclosure is directed to a medical imaging system. The medical imaging system may include a fluid source; imaging electronics; and an endoscope. The endoscope may include an elongate member having a proximal end and a distal end, and an imaging device disposed adjacent the distal end of the elongate member. The endoscope may further include a cable assembly electronically coupled to the imaging device and the imaging electronics. The cable assembly may include a plurality of conductors separated by insulation material to exclude fluid from being introduced between the plurality of conductors when the cable assembly is in a fluidic environment.

In various embodiments, the medical imaging system may include one or more of the following additional features: wherein the endoscope is a single-use endoscope; wherein the insulation material completely surrounds each of the plurality of conductors; wherein the cable assembly has a constant pitch between the plurality of conductors along the length of the cable assembly; wherein the endoscope further includes a port disposed at the distal end of the elongate member and in fluid communication with the fluid source; and wherein the imaging device includes an image sensor.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate embodiments of the invention and together with the description, serve to explain the principles of the invention.

### BRIEF DESCRIPTION OF THE DRAWINGS

FIG. 1 illustrates an exemplary medical imaging system including an endoscope and a control unit, according to an exemplary embodiment of the invention;

FIG. 2 is a cross-sectional view of the endoscope, according to an exemplary embodiment of the invention;

FIG. 3A is an exemplary electronic cable assembly for use with the endoscope, according to an exemplary embodiment of the invention; and

FIG. 3B is a cross-sectional view of the electronic cable assembly, according to an exemplary embodiment of the invention.

### DESCRIPTION OF THE EMBODIMENTS

Reference will now be made in detail to the present exemplary embodiment of the disclosure, an example of which is illustrated in the accompanying drawings. Wherever possible, the same reference numbers will be used throughout the drawings to refer to the same or like parts.

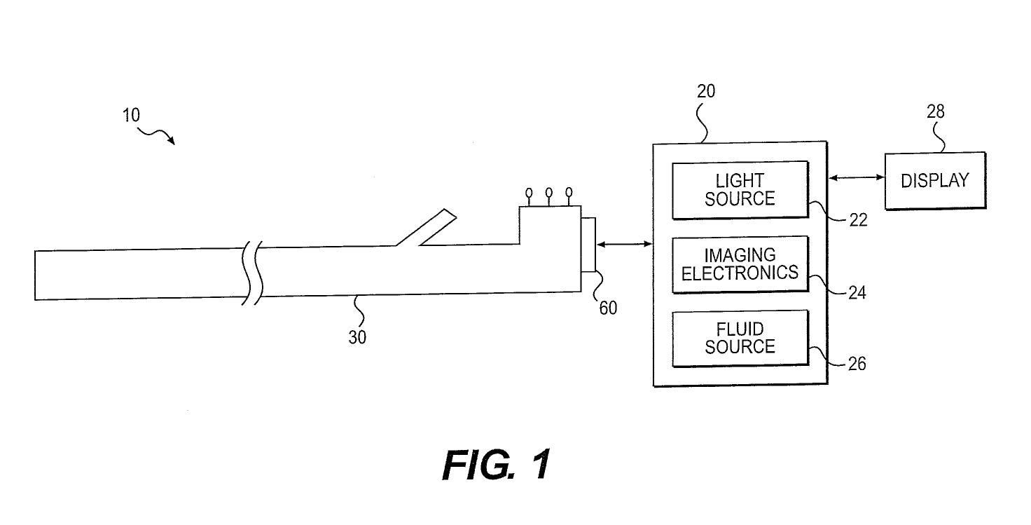

FIG. 1 illustrates an exemplary medical imaging system 10 and related components. The exemplary medical imaging system 10 may be used for therapeutic and/or diagnostic endoscopic procedures. The phrase “endoscopic procedure” is broadly used to indicate any medical procedure that may be performed by inserting an endoscope, ureteroscope, colonoscope, laparoscope, guide tube, catheter, or any such medical device into the body through any natural, surgical, percutaneous, or other opening in the body. The term “medical imaging system” is also used broadly to include all components and systems that may be used for the endoscopic procedure. In the exemplary embodiment, the components of medical imaging system 10 include a control unit 20, a display 28 connected to control unit 20, and an endoscope 30 connected to control unit 20 via a connector 60. Control unit 20 may include one or more peripheral support devices including a light source 22, imaging electronics 24, and a fluid source 26. The one or more peripheral support devices may be provided together in a console or may be separately provided.

As will be discussed in more detail below, endoscope 30 may be an electronic endoscope that allows a physician to view internal body cavities of a patient. In the exemplary embodiment, endoscope 30 may be sized for access and visualization of smaller ducts such as, for example, biliary and/or pancreatic ducts. The disclosed endoscope 30 may be sufficiently inexpensive to manufacture such that the endoscope 30 may be considered a single use, disposable item. However, those skilled in the art would recognize that the disclosure is applicable to any other endoscope sized for access and visualization of smaller ducts that are single-use such as, for example, catheters, imaging guidewires, and the like.

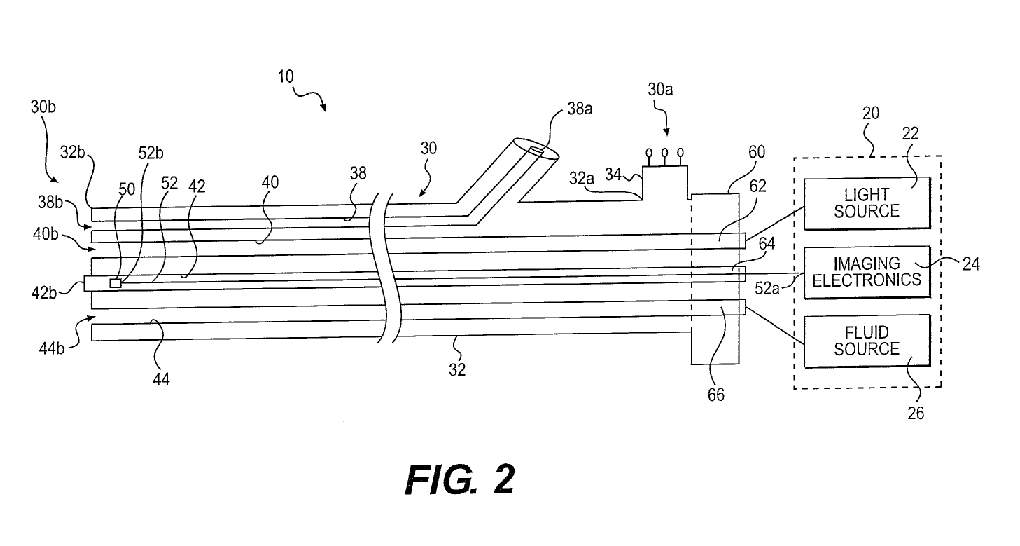

Referring to FIG. 2, endoscope 30 includes a proximal end 30a, a distal end 30b, and an elongate member 32 extending between proximal end 30a and distal end 30b. For purposes of this disclosure, “proximal” refers to the end closer to the device operator during use, and “distal” refers to the end further from the device operator during use.

A handle portion 34 is disposed at proximal end 30a of endoscope 30. Handle portion 34 is attached to connector 60. Connector 60 may have one or more lumens to connect endoscope 30 to the peripheral support devices disposed in control unit 20. In the embodiment shown in FIG. 2, the lumens 62, 64, and 66 are positioned adjacent to one another on the same side of connector 60. In an alternative embodiment, the lumens 62, 64, and 66 may be positioned on different sides of connector 60. Handle portion 34 may be any known, suitable handle having one or more valves, actuators, switches, or the like to control the delivery of fluid, light, air/gas, and/or images to and from control unit 20 and distal end 30b of endoscope 30.

Elongate member 32 extends distally from handle portion 34, and terminates at distal end 32b. Elongate member 32 may be a flexible tube, made from any suitable biocompatible material known to one of ordinary skill in the art and having sufficient flexibility to traverse a body cavity. Such materials may include, but are not limited to, rubber, silicon, synthetic plastic, stainless steel, metal-polymer composites, and metal alloys of nickel, titanium, copper cobalt, vanadium, chromium, aluminum, tantalum, and iron. In one embodiment, the material forming elongate member 32 may be a superelastic material such as nitinol, which is a nickel-titanium alloy. Elongate member 32 may have any cross-sectional shape and/or configuration, and may be any desired dimension that can be received in a catheter, guide, or body cavity. In one embodiment, elongate member 32 may have an outer diameter sized to ensure access to bile ducts, ureters, or tight strictures. It is contemplated that endoscope 30 and/or elongate member 32 may be steerable and may have areas of different flexibility or stiffness (e.g., to promote steerability).

Elongate member 32 may include one or more lumens extending substantially longitudinally (axially) within elongate member 32, and generally between proximal end 30a and distal end 30b of endoscope 30. In the exemplary embodiment depicted in FIG. 2, elongate member 32 includes four lumens: a working lumen 38, an illumination lumen 40, a visualization lumen 42, and an irrigation lumen 44. It should be understood, however, that elongate member 32 may include a greater or lesser number of lumens. For example, elongate member 32 may further include an aspiration lumen.

Working lumen 38 extends from a port 38a at a proximal end 32a of elongate member 32 and terminates at a working port 38b on distal end 32b of elongate member 32. Illumination lumen 40, irrigation lumen 44, and visualization lumen 42 extend distally from handle portion 34 and terminate at illumination port 40b, irrigation port 44b, and visualization port 42b, respectively, at distal end 32b of elongate member 32. The working lumen 38, illumination lumen 40, irrigation lumen 44, and visualization lumen 42 may have any suitable size, cross-sectional area, shape, and/or configuration. In some embodiments, these lumens may be extruded in elongate member 32.

Working port 38b and working lumen 38 may be configured to deliver an endoscopic instrument or device (not shown) to the work site adjacent distal end 30b of endoscope 30. The endoscopic instrument may be any tool that is configured to perform a desired function at the work site while being controlled by an actuation device from outside the body. Illumination port 40b may be configured to receive an illumination device (not shown) to illuminate tissue adjacent distal end 30b of endoscope 30. Illumination device may be connected to light source 22 (e.g., LEDs) by way of a fiber bundle (not shown) extending through illumination lumen 40 and a corresponding lumen 62 of connector 60. Alternatively, illumination device may be an fiber optic cable which may extend through illumination lumen 40 and corresponding lumen 62 of connector 60 to light source 22. In yet other embodiments, illumination device may be a light pipe conducted with a sheath. Irrigation port 44b may be configured to deliver fluids from the fluid source 26 to tissue adjacent distal end 30b of endoscope 30. More particularly, fluid may be circulated between fluid source 26 and irrigation port 44b of elongate member 32 by way of irrigation lumen 44 and a corresponding lumen 66 of connector 60.

An imaging device 50 is fitted within the visualization port 42b. Imaging device 20 may include a camera, lens, digital imaging chip, or other image receiving device, which may transmit signals in an analog or digital format. In an exemplary embodiment, imaging device 50 may include an image sensor such as a CMOS imaging sensor, CCD, or other solid state device, and one or more glass or polymeric lenses that produce electronic signals representative of an image of tissue in front of visualization port 42b. The image sensor may be a low light sensitive, low noise, CMOS color imager with VGA resolution or higher, such as SVGA, SXGA, or XGA. An electronic cable assembly 52 may be coupled to imaging device 50 and may extend proximally through visualization lumen 42 and the corresponding lumen 64 in connector 60 to imaging electronics 24. Electronic cable assembly 50 may have a distal end 52b coupled to imaging device 50 and a proximal end 52a coupled to imaging electronics 24. Image signals from imaging device 50 may be transmitted by way of electronic cable assembly 52 to be processed by imaging electronics 24 before being directed to display device 28. It is also contemplated that, in some devices, imaging electronics 24 may also direct a control signal to imaging device 50 via electronic cable assembly 52 to control various aspects of its operation.

In some embodiments, endoscope 30 may have small diameter lumens or utilize thin-wall lumens. In these embodiments, the walls between the visualization lumen 42 and the irrigation lumen 44 may be prone to processing defects and/or mechanical damage during a medical procedure. These defects may allow fluid from the irrigation lumen 44 to enter into the visualization lumen 42 (i.e., lumen cross-talk) and submerge electronic cable assembly 52 disposed in visualization lumen 42.

In conventional electronic cable assemblies, one or more conductors are captured and insulated in a single mass to form a cable assembly. In particular, a first thin layer of insulation material may be disposed on the conductors to provide top side insulation, and a second thin layer of insulation material may be disposed on the conductors to provide bottom side insulation. In this arrangement, an area may be formed between adjacent conductors which may be filled with fluid when the cable assembly is in a fluidic environment. As will be explained below, the fluid introduced into areas formed between adjacent conductors may increase the permittivity and the capacitance of these assemblies.

The capacitance of conventional cable assemblies may be calculated using the simplified equation for capacitance between parallel wires expressed below:

$$C = \frac{π ∈ l}{ln ( \frac{d}{2 a} + \sqrt{\frac{{d}^{2}}{4 {a}^{2}} - 1} )}$$

wherein C is the capacitance, a is the radius of the conductor, d is the distance between conductors, I is the conductor length, and ∈ is the permittivity of the material between the conductors. The above equation may be used to illustrate how fluid can affect the capacitance of the conventional cable assemblies. In the capacitance equation above, permittivity is defines as:

∈=∈ᵣ∈₀

wherein ∈ᵣ is the relative permittivity, or dielectric constant, of the material between conductors, and ∈₀ is a constant. As the dielectric constant of air is approximately 1 and that of water is 80, it may be understood that fluid between conductors increases the permittivity and therefore increases the capacitance of these cable assemblies. The increased capacitance may degrade the electronic signal, resulting in video degradation or failure.

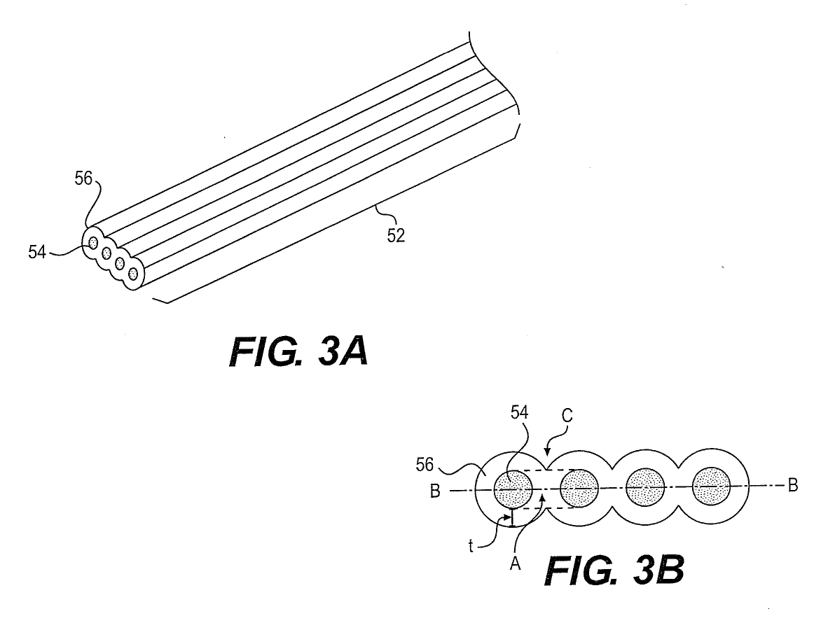

The disclosed electronic cable assembly 52 may be configured to exclude fluid from being introduced between the plurality of conductors when the electronic cable assembly 52 is in a fluidic environment. FIG. 3A is a schematic perspective view of an exemplary embodiment of the disclosed electronic cable assembly. The electronic cable assembly 52 illustrated in FIG. 3A may be a fine gage cable assembly including a plurality of longitudinally oriented conductors 54 encased in a shared layer of insulation material 56. The insulation material 56 may completely surround each conductor 54 and substantially fill an area between adjacent conductors 54.

In FIGS. 3A and 3B, four conductors 54 are shown as an exemplary number of conductors 54 disposed in an electronic cable assembly 52. However, it will be understood that any number of conductors may be disposed in the electronic cable assembly 52. In some embodiments, the conductors 54 may be configured and arrange as a single layer of conductors 54. Conductors 54 may be fine gage conductors between 30 to 50 gage, and, in some embodiments, 40 gage. The conductors 54 may be formed using a conductive, biocompatible material. Examples of suitable materials include metals, alloys, conductive polymers, conductive carbon, and any other biocompatible material known in the art, as well as combinations thereof. In the exemplary embodiments, conductors may have a substantially circular cross-section. It will be understood, however, that conductors may have a square or rectangular cross-section, or any other cross-section and/or configuration.

As illustrated in FIGS. 3A and 3B, insulation material 56 may form a sheath around each of the plurality of conductors 56. The sheath of each of the plurality of conductors may extend into an area and contact a sheath of an adjacent conductor 56. In this manner, insulation material 56 may substantially fill the area between adjacent conductors 56. The area between adjacent conductors is depicted in FIG. 3B. As shown in FIG. 3B, the area “A” may have a length corresponding to the length between adjacent conductors and a width substantially corresponding to a diameter of the conductors. In some embodiments, the area A is completely filled by the insulation material 56. In some additional embodiments, the insulation material 56 may be minimized in other areas between conductors 54 outside area A (i.e., in a direction transverse to line B-B) to form depressions or grooves C between adjacent conductors 54, while still maintaining a consistent thickness t of insulation material 56 about the circumference of each conductor 54. This may minimize the profile of the cable assembly 52, and consequently, the manufacturing expenses.

Insulation material 56 may be formed using any non-conductive, biocompatible material. Examples of suitable materials include rubber, silicone, polyurethane, ethylene, tertrafluoroethylene, polytetrafluoroethylene, polydimethylsiloxane, and any other non-conductive biocompatible material known in the art. In some embodiments, a single layer of insulation material 56 may encase the plurality of conductors 54. It is contemplated that, in at least some embodiments, the thickness of the insulation material 56 disposed between adjacent conductors may be approximately equal to the thickness of the insulation material disposed around each conductor 54. In some embodiments, the thickness of the insulation (i.e., in a direction traverse to line B-B) may be greater than the insulation disposed around each conductor 54. The same or different insulation material may be disposed around each conductor 54 and between conductors 54 in area A.

It is contemplated that weakened regions may be formed in one or more desired locations in the insulation to facilitate bending or folding, or to facilitate separation of one or more portions of electronic cable assembly 52. For example, in some embodiments weakened regions may be formed in insulation material 56 along the longitudinal length of electronic cable assembly 52 to separate one or more conductors 54 from electronic cable assembly 52.

It is further contemplated that insulation material 56 may be removed or ablated at selected locations to expose one or more underlying conductors 54 for electrically coupling the one or more exposed conductors 54 to imaging device 50 and/or imaging electronics 24. In other embodiments, the entire insulation material 56 may be removed from ends 52a and/or 52b to facilitate electrical coupling to the imaging device 50 and/or imaging electronics 24.

In some additional embodiments, electronic cable assembly 52 may further include a shielding, which may encapsulate conductors 54 to minimize noise between conductors 54 and the environment. In those embodiments, the shielding may be disposed between two layers of insulation material 56 that form a sheath around the conductors 54. In alternative embodiments, the shielding may be disposed about each individual conductor. The shielding may be composed of any known conducting material including metal alloys and conductive polymers.

The disclosed electronic cable assembly 52 may be formed by an extrusion process that ensures that conductors 54 are encased in a shared insulation material 56. In a suitable extrusion process, plastic pellets, typically at room temperature, may be placed into an extruder and heated above the melting temperature of the plastic. The extruder may also pressurize the plastics when in the molten stage. Once molten and at a high pressure, the plastic melt may be forced through a die that has been cut to provide the desired profile of the cable assembly. The conductors 54 may also be introduced into the die, as is known in the art. Once the melt has been forced through the die, the melt may be cooled, preferably in a bath, to harden the plastic into the required shape and profile.

The extruded electronic cable assembly 52 process may provide certain benefits. For example, the extrusion process may produce cable assemblies of the long lengths required for endoscopes while maintaining a constant pitch and remaining inexpensive. A constant pitch may control the impedance between conductors so as to prevent electrical signal degradation. Furthermore, a controlled pitch may ensure that the location of each conductor 54 is known when the insulation is stripped and the cable assembly is attached to imaging device 50 and the proximal electronics 24. This, in turn, may reduce manufacturing costs. It is understood, however, that the disclosed cable assembly 52 may be formed by any other process including, for example, molding, casting, extrusion dip coating, and the like. For example, insulation may be injection molded around each conductor 54. Alternatively, portions of the cable assembly 52 may be formed separately and joined together with heat or ultrasonic welding.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A cable assembly for an electronic medical device, the cable assembly comprising:
a plurality of conductors each defining a longitudinal axis, wherein the plurality of conductors are spaced so that the longitudinal axes of the plurality of conductors are parallel; and
a layer of insulation material completely surrounding each of the plurality of conductors and extending between adjacent conductors to substantially fill areas between adjacent conductors along a length of the cable assembly, wherein at least one conductor is electrically coupled to electronics at a distal portion of the electronic medical device.

2. The cable assembly of claim 1, wherein the layer of insulation material completely fills areas between adjacent conductors along the length of the cable assembly.

3. The cable assembly of claim 1, wherein the layer of insulation material is arranged to exclude fluid from being introduced between the plurality of conductors when the cable assembly is in a fluidic environment.

4. The cable assembly of claim 1, wherein the longitudinal axes of the plurality of conductors are aligned in a single plane.

5. The cable assembly of claim 1, wherein a portion of at least one conductor is exposed.

6. A medical device, comprising:
an elongate member having a distal end, a first lumen, and a second lumen;
a port defined in the distal end of the elongate member, the fluid port being in communication with the first lumen for delivering fluid;
an imaging device at the distal end of the elongate member; and
a cable assembly electrically coupled to the imaging device and extending proximally in the second lumen, wherein the cable assembly includes two or more conductors and a single layer of insulation, wherein the insulation substantially fills areas between adjacent conductors.

7. The medical device of claim 6, wherein the single layer of insulation completely surrounds each conductor of the two or more conductors.

8. The medical device of claim 6, wherein the single layer of insulation material is arranged to exclude fluid from being introduced between the two or more conductors when the cable assembly is in a fluidic environment.

9. The medical device of claim 6, wherein the longitudinal axes of the two or more conductors are parallel and disposed in a single plane.

10. The medical device of claim 6, wherein the cable assembly has a length, and wherein each of the two or more conductors is arranged longitudinally along the length of the cable assembly.

11. The medical device of claim 6, wherein the two or more conductors have a constant pitch along the length of the cable assembly.

12. The medical device of claim 6, wherein the conductors are fine gage conductors.

13. The medical device of claim 6, wherein the imaging device is configured to produce an image signal in an analog format.

14. The medical device of claim 6, wherein the single layer of insulation material completely fills areas between adjacent conductors.

15. A medical imaging system, comprising:
a fluid source;
imaging electronics; and
an endoscope, comprising:
an elongate member having a proximal end and a distal end;
an imaging device disposed adjacent the distal end of the elongate member; and
a cable assembly electronically coupled to the imaging device and the imaging electronics, wherein the cable assembly includes a plurality of conductors separated by insulation material to exclude fluid from being introduced between the plurality of conductors when the cable assembly is in a fluidic environment.

16. The medical imaging system of claim 15, wherein the endoscope is a single-use endoscope.

17. The medical imaging system of claim 15, wherein the insulation material completely surrounds each of the plurality of conductors.

18. The medical imaging system of claim 15, wherein the cable assembly has a constant pitch between the plurality of conductors along the length of the cable assembly.

19. The medical imaging system of claim 15, wherein the endoscope further includes a port disposed at the distal end of the elongate member and in fluid communication with the fluid source.

20. The medical imaging system of claim 15, wherein the imaging device includes an image sensor.
